(19) United States
(12) Patent Application Publication
(10) Pub. No.: US 2008/0087878 A1
(43) Pub. Date: Apr. 17, 2008

(54) USE OF PERYLENE DIIMIDE DERIVATIVES AS AIR-STABLE N-CHANNEL ORGANIC SEMICONDUCTORS

(75) Inventors: Martin KOENEMANN, Mannheim (DE); Peter Erk, Frankenthal (DE); Marcos Gomez, Santa Barbara, CA (US); Zhenan Bao, Stanford, CA (US); Mang-Mangike (Mike) Ling, Santa Clara, CA (US)

Correspondence Address: OBLON, SPIVAK, MCCLELLAND MAIER & NEUSTADT, P.C., 1940 DUKE STREET, ALEXANDRIA, VA, 22314, US

(73) Assignees: BASF Akiengesellschaft, Ludwigshafen (DE); Stanford University, Stanford, CA (US)

(21) Appl. No.: 11/550,250

(22) Filed: Oct. 17, 2006

(51) Int. Cl. H01L 29/08; C07D 471/02; H01L 51/40

(52) U.S. Cl. 257/40; 438/99; 546/37; 257/E51.024

(57) ABSTRACT

The present invention relates to the use of of perylene diimide derivatives as air-stable n-type organic semiconductors.

### BACKGROUND OF THE INVENTION

1. Field of the Invention

The present invention relates to the use of perylene diimide derivatives as air-stable n-type organic semiconductors.

2. Description of the Related Art

In the field of microelectronics there is a constant need to develop smaller device elements that can be reproduced conveniently and inexpensively at a lowest possible failure rate. Modern digital integrated circuits are based on field-effect transistors (FET), which rely on an electric field to control the conductivity of a “channel” in a semiconductor material. Organic field-effect transistors (OFET) allow the production of flexible or unbreakable substrates for integrated circuits having large active areas. As OFETs enable the production of complex circuits, they have a wide area of potential application (e.g. in driver circuits of pixel displays). A thin film transistor (TFT) is a special kind of field effect transistor made by depositing thin films for the metallic contacts, semiconductor active layer, and dielectric layer. The channel region of a TFT is a thin film that is deposited onto a substrate (e.g. glass for application of TFTs in liquid crystal displays).

A major class of semiconductors for integrated circuits (IC) are complementary metal-oxide semiconductors (CMOS). CMOS chips are still omnipresent in microprocessors, microcontrollers, static RAM and other digital logic circuits. Over the past few years great efforts were made to synthesize high performance n-channel organic semiconductors to replace MOSFETs (metal oxide semiconductor field-effect transistors) in the production of integrated circuits. Examples of organic semiconducting compounds are C₆₀ and its derivatives, copper hexadecafluoro phthalocyanine (F₁₆CuPc), perylenes and perylene derivatives, oligothiophenes and oligothiophene derivatives. Apart from good electron mobility, an important property of organic semiconducting compounds is a good air resistance. A basic design principle to obtain air-stable n-type semiconductors has been to incorporate strong electron-withdrawing groups, such as fluorine groups. However, this usually requires a complicated synthesis which makes the use of said materials uneconomic. US 2002/0164835 A1 (U.S. Pat. No. 7,026,643 B2) teaches the use of N,N′-perylene-3,4,9,10-tetracarboxylic diimide as n-type semiconductor material. It is disclosed in very general terms that also perylene tetracarboxylic diimides with linear chains of 4 to 18 saturated atoms bound to the imide nitrogen atoms are suitable as n-type semiconductors. In particular N,N′-di-(n-octyl) perylene-3,4,9,10-tetracarboxylic diimide and N,N′-di(n-1H,1H perfluorooctyl) perylene-3,4,9,10-tetracarboxylic diimide are named without any evidence by an example.

US 2003/0181721 A1 (Wuerthner) discloses tetra-substituted perylenetetracarboxylic diimides of the formula

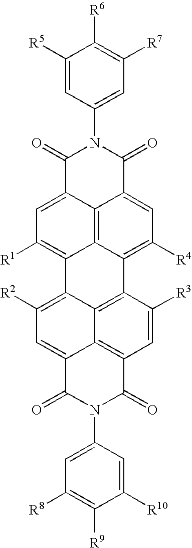

where

R¹, R², R³ and R⁴ are independently hydrogen, chlorine, bromine, substituted or unsubstituted aryloxy, arylthio, arylamino, hetaryloxy or hetarylthio,

R⁵, R⁶, R⁷, R⁸, R⁹ and R¹⁰ are independently hydrogen or long-chain alkyl, alkoxy or alkylthio with the proviso that at least four of these radicals are not hydrogen.

It is also mentioned in very general terms that such perylimides are useful for electronics, optoelectronics and photonic applications such as charge transport materials in luminescent diodes and photovoltaic diodes, photoconductors and transistors. This document also does not teach a method for the production of OFETs.

J. Ostrick, A. Dodabalapur, L. Torsi, A. J. Lovinger, E. W. Kwock, T. M. Miller, M. Galvin, M. Berggren, and H. E. Katz disclose in J. Appl. Phys. 81 (10), 1997, 6804-6808 the electron transport properties of perylenetetracarboxylic dianhydride.

D. J. Gundlach, K. P. Pernstich, G. Wilckens, M. Grüter, S. Haas, and B. Batlogg report in J. Appl. Phys. 98, 064502 (2005), on n-channel organic thin-film transistors (OTFTS) using N,N′-ditridecylperylene-3,4,9,10-tetracarboxylic diimide as semiconductor material.

M. Hiramoto, K. Ihara, H. Fukusumi, and M. Yokoyama describe in J. Appl. Phys. 78 (12), 1995, 7153-7157 the effects of purification by reactive sublimation technique and bromine doping on the photovoltaic properties of n-type perylene pigment films. N,N′-dimethylperylene-3,4,9,10-tetracarboxylic diimide was purified by sublimation and exposed to Br₂ gas. and afterwards the photovoltaic properties and current-voltage characteristics were measured.

R. J. Chesterfield, J. C. McKeen, C. R. Newman, P. C. Ewbank, D. A. Da Silva Filho, J. L. Brédas, L. L. Miller, K. R. Mann, and C. D. Frisbie describe in J. Phys. Chem. B 2004, 108,19281-19292 organic thin film transistors based on N-alkyl perylene diimides of the formula

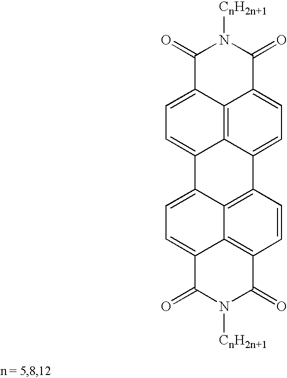

G. Horowitz, F. Kouki, P. Spearman, D. Fichou, C. Nogues, X. Pan, and F. Gamier describe in Adv. Mater. 1996, 8, No. 3, 242-244 photovoltaic diodes and FET with N,N′-diphenylperylene-3,4,9,10-tetracarboxylic diimide.

J. Locklin, D. Li, S. C. B. Mannsfeld, E.-J. Borkent, H. Meng, R. Advincula, and Z. Bao report in Chem. Mater. 2005, 17, 3366 3374 on organic thin film transistors based on cyclohexyl-substituted organic semiconductors, inter alia N,N′-dicyclohexylperylene-3,4,9,10-tetracarboxylic diimide.

M. J. Ahrens, M. J. Fuller and M. R. Wasielewski describe in Chem. Mater. 2003, 15, p. 2684-2686 cyano-substituted perylene-3,4-dicarboximides and perylene-3,4,9,10-bis(dicarboximides) and the use thereof as chromophoric oxidants, e.g. for photonic and electronic.

B. A. Jones et al. describe in Angew. Chem. 2004, 116, S. 6523-526 the use of dicyano perylene-3,4,9,10-bis(dicarboximides) as n-type semiconductors.

US 2005/0176970 A1 discloses substituted perylene-3,4-dicarboximides and peryiene-3,4,9,10-bis(dicarboximides) as n-type semiconductors.

Unpublished U.S. patent applcation Ser. No. 11/417,149 describes organic-field effect transistors, on the basis of an n-type organic semiconducting compound of the formula I

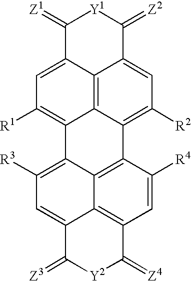

wherein

R¹, R², R³ and R⁴ are independently hydrogen, chlorine or bromine, with the proviso that at least one of these radicals is not hydrogen,

Y¹ is O or NR^{a}, wherein R^{a} is hydrogen or an organyl residue,

Y² is O or NR^{b}, wherein R^{b} is hydrogen or an organyl residue,

Z¹, Z², Z³ and Z⁴ are O,

where, in the case that Y¹ is NR^{a}, one of the residues Z¹ and Z² may be a NR^{c} group, where R^{a} and R^{c} together are a bridging group having 2 to 5 atoms between the terminal bonds,

where, in the case that Y² is NR^{b}, one of the residues Z³ and Z⁴ may be a NR^{d} group, where R^{b} and R^{d} together are a bridging group having 2 to 5 atoms between the terminal bonds.

Unpublished European patent application 06007415

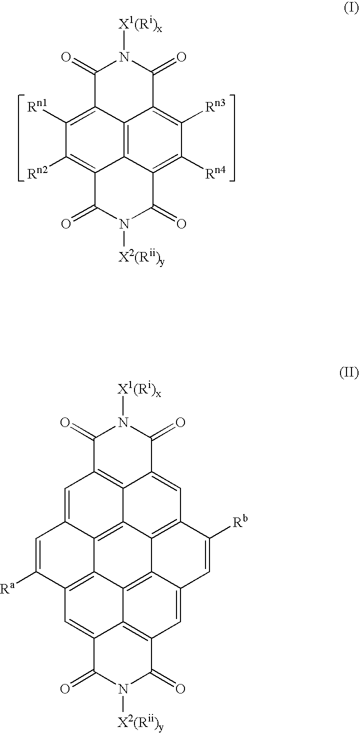

where

n is 1, 2, 3 or 4,

x and y are an integer of 2 to 6,

Rⁿ¹, Rⁿ², Rⁿ³ and Rⁿ⁴ for n=1 or 2 are selected from H, F, Cl, Br and CN and for n=3 or 4 are selected from H, F, Cl und Br,

R^{a} and R^{b} are H or alkyl,

X¹ is an (x+1)-valent residue,

X² is an (y+1)-valent residue,

Rⁱ und Rⁱⁱ are independently selected from C₄-C₃₀ alkyl, that can be interrupted by one or more than one oxygen atoms,

as n-type semiconductor for OFETs or solar cells.

F. Nolde, W. Pisula, S. Müller, C. Kohl, and K. Müllen describe in Chem. Mater. 2006, 18, 3715-3725 the synthesis and self-organization of core-extended perylene tetracarboxdiimides with branched alkyl substituents of the formulae

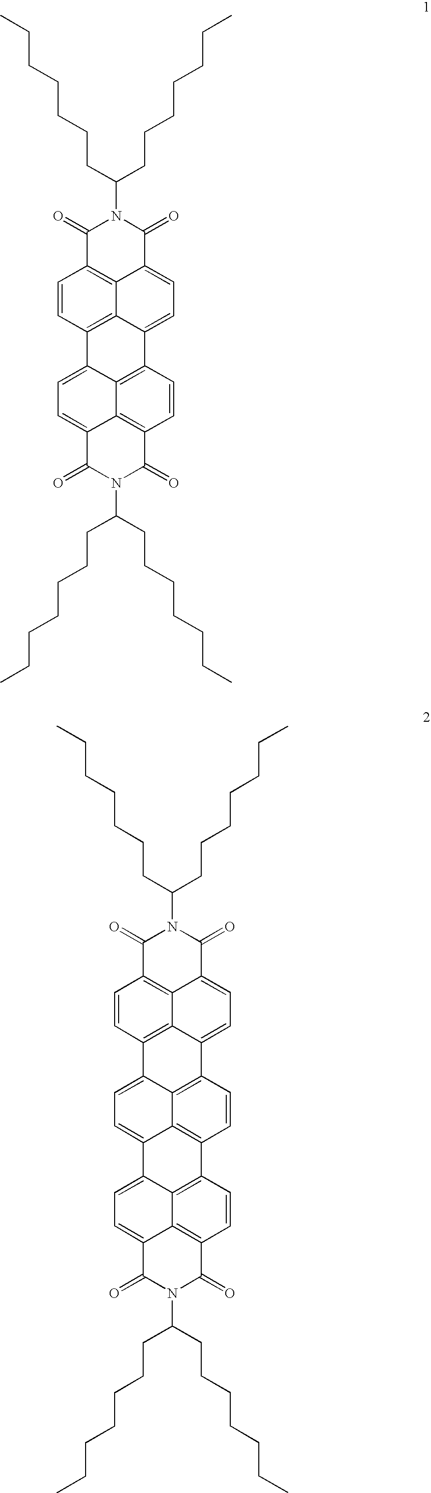

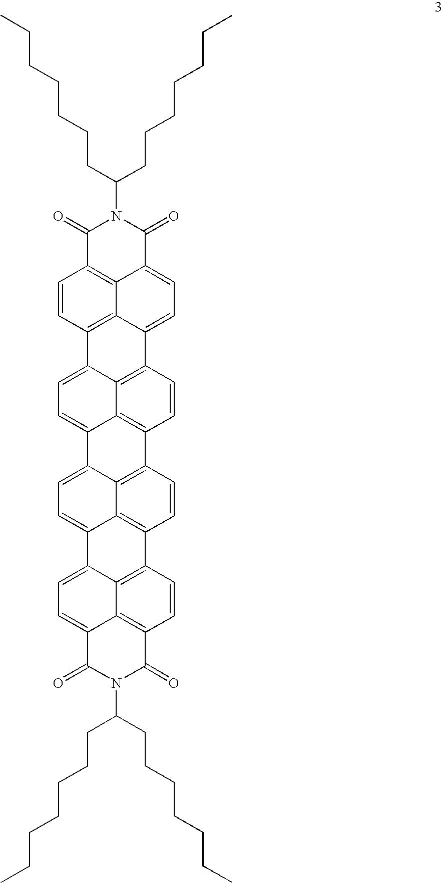

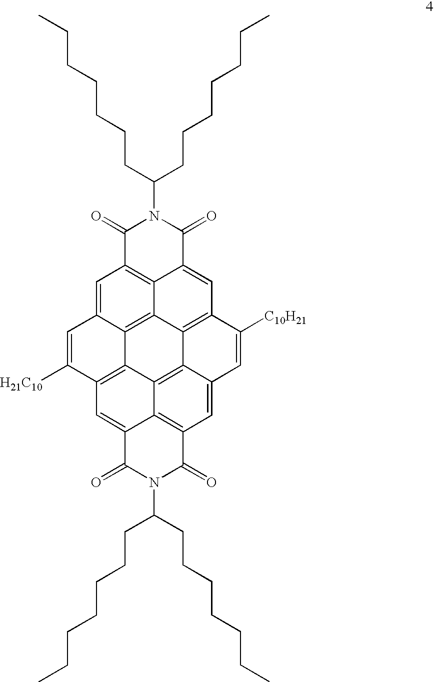

It was now surprisingly found that peryiene diimide derivatives without strong electron withdrawing groups and with linear C₁-C₄ alkyl groups, optionally carrying a terminal cyclic group, bound to the imide nitrogen atoms have a good transistor performance and good air-stability.

### SUMMARY OF THE INVENTION

In a first aspect, the invention provides a method for producing an organic field-effect transistor, comprising the steps of:
a) providing a substrate comprising a gate structure, a source electrode and a drain electrode located on the substrate, and
b) applying at least one compound of the formula I

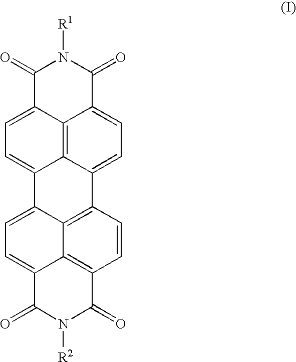

wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4,
as n-type organic semiconducting compound to the area of the substrate where the gate structure, the source electrode and the drain electrode are located.

According to a special embodiment, said method comprises the step of depositing on the surface of the substrate at least one compound (C1) capable of binding to the surface of the substrate and of binding at least one compound of the formula (I).

In a further aspect, the invention provides an organic field-effect transistor comprising:
a substrate,
a gate structure, a source electrode and a drain electrode located on the substrate, and
at least one compound of the formula (I) as n-type organic semiconducting compound at least on the area of the substrate where the gate structure, the source electrode and the drain electrode are located.

In a further aspect, the invention provides an organic field-effect transistor obtainable by a method, comprising the steps of:
a) providing a substrate comprising a gate structure, a source electrode and a drain electrode located on the substrate, and
b) applying at least one compound of the formula (I) as n-type organic semiconducting compound to the area of the substrate where the gate structure, the source electrode and the drain electrode are located.

In a further aspect, the invention provides a method for producing a substrate comprising a pattern of n-type organic field-effect transistors, wherein at least part of the transistors comprise at least one compound of the formula (I) as n-type organic semiconducting compound.

In a further aspect, the invention provides a substrate comprising a pattern of n-type organic field-effect transistors wherein at least part of the transistors comprise a compound of the formula (I) as n-type organic semiconducting compound.

In a further aspect, the invention provides a method for producing an electronic device comprising the step of providing on a substrate a pattern of organic field-effect transistors, wherein at least part of the transistors comprise at least one compound of the formula (I) as n-type organic semiconducting compound.

In a further aspect, the invention provides an electronic device comprising on a substrate a pattern of organic field-effect transistors, wherein at least part of the transistors comprise at least one compound of the formula (I) as n-type organic semiconducting compound.

The method according to the invention can be used to provide a wide variety of devices. Such devices may include electrical devices, optical devices, optoelectronic devices (e.g. semiconductor devices for communications and other applications such as light emitting diodes, electroabsorptive modulators and lasers), mechanical devices and combinations thereof. Functional devices assembled from transistors obtained according to the method of the present invention may be used to produce various IC architectures. Further, at least one compound of the formula (I) may be employed in conventional semiconductor devices, such as diodes, light-emitting diodes (LEDs), inverters, sensors, and bipolar transistors. One aspect of the present invention includes the use of the method of the invention to fabricate an electronic device from adjacent n-type and/or p-type semiconducting components. This includes any device that can be made by the method of the invention that one of ordinary skill in the art would desirably make using semiconductors. Examples of such devices include, but are not limited to, field effect transistors (FETs), bipolar junction transistors (BJTs), tunnel diodes, modulation doped superlattices, complementary inverters, light-emitting devices, light-sensing devices, biological system imagers, biological and chemical detectors or sensors, thermal or temperature detectors, Josephine junctions, nanoscale light sources, photodetectors such as polarization-sensitive photodetectors, gates, inverters, AND, NAND, NOT, OR, TOR, and NOR gates, latches, flip-flops, registers, switches, clock circuitry, static or dynamic memory devices and arrays, state machines, gate arrays, and any other dynamic or sequential logic or other digital devices including programmable circuits.

A special type of electronic device in an inverter. In digital logic an inverter is a logic gate which inverts the digital signal driven on its input. It is also called NOT gate. The truth table of the gate is as follows: input 0=output 1; input 1=output 0. In practice, an inverter circuit outputs a voltage representing the opposite logic-level as its input. Digital electronics are circuits that operate at fixed voltage levels corresponding to a logical 0 or 1. An inverter circuit serves as the basic logic gate to swap between those two voltage levels. Implementation determines the actual voltage, but common levels include (0, +5V) for TTL circuits. Common types include resistive-drain, using one transistor and one resistor; and CMOS (complementary metal oxide semiconductor), which uses two (opposite type) transistors per inverter circuit. The performance quality of a digital inverter can be measured using the Voltage Transfer Curve (VTC), i.e. a plot of input vs. output voltage. From such a graph, device parameters including noise tolerance, gain, and operating logic-levels can be obtained. Ideally, the voltage transfer curve (VTC) appears as an inverted step-function (i.e. precise switching between on and off) but in real devices, a gradual transition region exists. The slope of this transition region is a measure of quality: the steeper (close to infinity) the slopes the more precise the switching. The tolerance to noise can be measured by comparing the minimum input to the maximum output for each region of operation (on/off). The output voltage VOH can be a measure of signal driving strength when cascading many devices together. The digital inverter is considered the base building block for all digital electronics. Memory (1 bit register) is built as a latch by feeding the output of two serial inverters together. Multiplexers, decoders, state machines, and other sophisticated digital devices all rely on inverter.

In a further aspect the invention provides an inverter comprising at least one compound of the formula I as n-type organic semiconducting compound. A special embodiment are CMOS inverter comprising two (opposite type) transistors. For high speed CMOS circuits, it is highly desirable that both p- and n-channel semiconductors have similar good mobilities. For p-channel transistors, there are a number of candidates with mobility greater than 0.1 cm²Vs, e.g. pentacene. Now it was surprisingly found that the compounds of the formula I can be advantageously employed as n-type semiconductors in inverters.

In a further aspect the invention provides the use of at least one compound of the formula (I) as n-type semiconductors. The compounds of the formula (I) are especially advantageous as n-type semiconductors for organic field-effect transistors, organic solar cells and organic light-emitting diodes (OLEDs).

In a further aspect the invention provides a method for producing a crystalline compound of the formula (I) as an n-type organic semiconducting compound comprising subjecting at least one compound of the formula (I) to a physical vapor transport (PVT).

### BRIEF DESCRIPTION OF THE DRAWINGS

FIGS. 1a and 1b show current-voltage characteristics of TFTs with N,N′-Bis(2-phenylethyl)perylene-3,4:9,10-bis(dicarboximide) (BPE-PTCDI).

FIG. 2 shows the out-of-plane XRD patterns of 40 nm BPE-PTCDI thin film deposited at a temperature of 150° C. on a plain substrate and substrates where the surface was treated with n-(octadecyl)trimethoxysilane (OTS) and hexamethyidisilazane (HMDS).

FIG. 3 shows air-stability measurements of BPE-PTCDI TFTs (2a: charge carrier mobility as a function of time, 2b: on/off ratio as a function of time).

FIG. 4 shows the atomic force microscope (AFM) images of 45 nm BPE-PTCDI thin films on substrates treated with n-(octadecyl)trimethoxysilane for various substrate temperatures (room temperature, 125° C., 150° C. and 200° C.) during thin film deposition.

FIG. 5 shows the out-of-plane XRD patterns of 40 nm BPE-PTCDI thin film deposited at a temperature of 125° C. on a substrates where the surface was treated with n-(octadecyl)trimethoxysilane (OTS).

FIG. 6 shows the cyclic voltammetry of BPE-PTCDI.

FIG. 7 shows the structure of an inverter structure comprising BPE-PTCDI as n-type transistor and pentacene as p-type transistors.

FIGS. 8(a) and 8(b) show typical current-voltage characteristics of pentacene and BPE-PTCDI.

FIG. 9 shows that the highest gain for a BPE-PTCDI inverter for V_{dd}=50 V is about 10.5, the noise margin is 8.5 V and the output voltage swing is about 46 V.

FIG. 10 shows the hysteresis for BPE-PTCDI.

### DETAILED DESCRIPTION OF PREFERRED EMBODIMENTS OF THE INVENTION

The term “C₁-C₄-alkyl” embraces straight-chain and branched alkyl groups. These groups are in particular, methyl, ethyl, n-propyl, isopropyl, n-butyl, isobutyl, sec-butyl, tert-butyl. This applies also to all alkyl moieties in alkoxy, alkylamino, dialkylamino, alkylthio, etc.

C₁-C₄-alkylene embraces straight-chain and branched hydrocarbon chains with 1 to 4 carbon atoms, in particular CH₂, CH₂CH₂, CH(CH₃), CH₂CH₂CH₂, CH(CH₃)CH₂, CH₂CH(CH₃), CH₂CH₂CH₂CH₂, CH(CH₃)CH₂CH₂, CH₂CH(CH₃)CH₂, CH ₂CH₂CH(CH₃), CH(C₂H₅)CH₂, CH₂CH(C₂H₅).

For the purposes of the present invention, the term “cycloalkyl” embraces both substituted and unsubstituted cycloalkyl groups, preferably C₃-C₈-cycloalkyl groups like cyclopropyl, cyclobutyl, cyclopentyl, cyclohexyl, cycloheptyl or cyclooctyl, in particular C₅-C₈-cycloalkyl. Substituted cycloalkyl groups can carry, for example, 1, 2, 3, 4, 5 or more than 5 substituents which are preferably selected independently of alkyl and substituents as defined above for “alkyl”. Substituted cycloalkyl groups carry preferably one or more, e.g. 1, 2, 3, 4 or 5, C₁-C₆-alkyl groups.

Examples of preferred cycloalkyl groups are cyclopropyl, cyclobutyl, cyclopentyl, 2- and 3-methylcyclopentyl, 2- and 3-ethylcyclopentyl, cyclohexyl, 2-, 3- and 4-methylcyclohexyl, 2-, 3- and 4-ethylcyclohexyl, 3- and 4-propylcyclohexyl, 3- and 4-isopropylcyclohexyl, 3- and 4-butylcyclohexyl, 3- and 4-sec.-butylcyclohexyl, 3- and 4-tert.-butylcyclohexyl, cycloheptyl, 2-, 3- and 4-methylcycloheptyl, 2-, 3- and 4-ethylcycloheptyl, 3- and 4-propylcycloheptyl, 3- and 4-isopropylcycloheptyl, 3- and 4-butylcycloheptyl, 3- and 4-sec.-butylcycloheptyl, 3- and 4-tert.-butylcycloheptyl, cyclooctyl, 2-, 3-, 4- and 5-methylcyclooctyl, 2-, 3-, 4- and 5-ethylcyclooctyl, 3-, 4- and 5-propylcyclooctyl.

For the purposes of the present invention, the term “cycloalkenyl” embraces unsubstituted and substituted monounsaturated hydrocarbon groups having 3 to 8, preferably 5 to 6, carbon ring members, such as cyclopenten-1-yl, cyclopenten-3-yl, cyclohexen-1-yl, cyclohexen-3-yl, cyclohexen-4-yl and the like. Suitable substituents for cycloalkenyl are the same as those mentioned above for cycloalkyl.

The term “bicycloalkyl” preferably embraces bicyclic hydrocarbon groups having 5 to 10 carbon atoms such as bicyclo[2.2.1]hept-1-yl, bicyclo[2.2.1]hept-2-yl, bicyclo[2.2.1]hept-7-yl, bicyclo[2.2.2]oct-1-yl, bicyclo[2.2.2]oct-2-yl, bicyclo[3.3.0]octyl, bicyclo[4.4.0]decyl and the like.

For the purposes of the present invention, the term “aryl” embraces monocyclic or polycyclic aromatic hydrocarbon radicals which may be unsubstituted or unsubstituted. Aryl is preferably unsubstituted or substituted phenyl, naphthyl, indenyl, fluorenyl, anthracenyl, phenanthrenyl, naphthacenyl, chrysenyl, pyrenyl, etc., and in particular phenyl or naphthyl. Aryl, when substituted, may carry—depending on the number and size of the ring systems—one or more (e.g. 1, 2, 3, 4, 5 or more than 5) substituents which are preferably selected independently of one another from among alkyl, alkoxy, thioalkyl, cycloalkyl, heterocycloalkyl, aryl, hetaryl, halogen, hydroxy, mercapto, COOH, carboxylate, SO₃H, sulfonate, NE¹E², nitro and cyano, where E¹ und E², independently of one another, are hydrogen, alkyl, cycloalkyl, heterocycloalkyl, aryl or hetaryl. Aryl is in particular phenyl which, when substituted, generally may carry 1, 2, 3, 4 or 5, preferably 1, 2 or 3, substituents.

For the purposes of the present invention heterocycloalkyl embraces nonaromatic, unsaturated or fully saturated, cycloaliphatic groups having generally 5 to 8 ring atoms, preferably 5 or 6 ring atoms, in which 1, 2 or 3 of the ring carbon atoms are replaced by heteroatoms selected from oxygen, nitrogen, sulfur, and a group —NR³—,said cycloaliphatic groupsfurther being unsubstituted or substituted by one or more—for example, 1, 2, 3, 4, 5 or 6 —C₁-C₆ alkyl groups. Examples that may be given of such heterocycloaliphatic groups include pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, dihydrothien-2-yl, tetrahydrofuranyl, dihydrofuran-2-yl, tetrahydropyranyl, 1,2-oxazolin-5-yl, 1,3-oxazolin-2-yl, and dioxanyl.

For the purposes of the present invention heteroaryl embraces substituted or unsubstituted, heteroaromatic, monocyclic or polycyclic groups, preferably the groups pyridyl, quinolinyl, acridinyl, pyridazinyl, pyrimidinyl, pyrazinyl, pyrrolyl, imidazoiyl, pyrazolyl, indolyl, purinyl, indazolyl, benzotriazolyl, 1,2,3-triazolyl, 1,3,4-triazolyl, and carbazolyl, which, when substituted, can carry generally 1, 2 or 3 substituents. The substituents are selected from C₁-C₆ alkyl, C₁-C₆ alkoxy, hydroxyl, carboxyl, halogen and cyano.

5- to 7-membered heterocycloalkyl or heteroaryl radicals bonded by a nitrogen atom and optionally containing further heteroatoms are, for example, pyrrolyl, pyrazolyl, imidazolyl, triazolyl, pyrrolidinyl, pyrazolinyl, pyrazolidinyl, imidazolinyl, imidazolidinyl, pyridinyl, pyridazinyl, pyrimidinyl, pyrazinyl, triazinyl, piperidinyl, piperazinyl, oxazolyl, isooxazolyl, thiazolyl, isothiazolyl, indolyl, quinolinyl, isoquinolinyl or quinaldinyl. Halogene is fluorine, chlorine, bromine or iodine.

In a preferred embodiment R¹ and R² are selected from cyclopropane, cyclobutane and cyclopentane.

In a preferred embodiment R¹ is selected from CH₂—R^{a}, CH₂CH₂—R^{a}, CH₂CH₂CH₂—R^{a} and CH₂CH₂CH₂CH₂—R^{a}. In a preferred embodiment R² is selected from CH₂—R^{b}, CH₂CH₂—R^{b}, CH₂CH₂CH₂—R^{b} and CH₂CH₂CH₂CH₂—R^{b}.

Preferably R^{a} and R^{b} are selected from

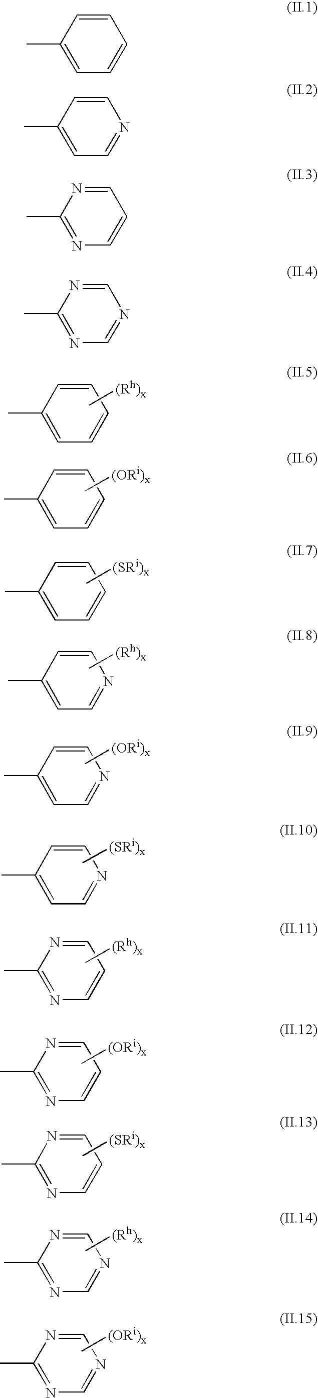

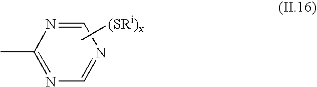

wherein
the residues R^{h} in formulae II.5, II.8, II.11 and II.14 are selected independently of one another from C₁-C₃-alkyl, C₁-C₃-fluoroalkyl, fluorine, chlorine, bromine, NE¹E², nitro and cyano, where E¹ und E², independently of one another, are hydrogen, alkyl, cycloalkyl, heterocycloalkyl, aryl or hetaryl,
the residues Rⁱ in formulae II.6, II.7, II.9, II.10, II. 12, II.13, II.15 and II.16 are selected independently of one another from C₁-C₃-alkyl,
x in formulae II.5, II6 and II.7 is 1, 2, 3, 4 or 5,
   in formulae II.8, II.9 and II.10 is 1, 2, 3 or 4,
   in formulae II.11, II.12 and II.13 is 1, 2 or 3,
   in formulae II.14, II.15 and II.16 is 1 or 2.

Preferably, n is 1 or 2.

In a preferred embodiment, R¹ and R² have the same meaning.

Especially preferred are compounds of the formulae:

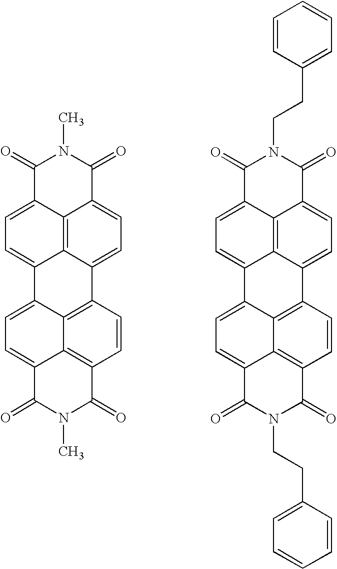

### Step a)

Step a) of the method for producing an OFET comprises providing a substrate with at least one preformed transistor site located on the substrate. It will be understood that when an element such as a layer, region or substrate is referred to as being “on” another element, it can be directly on the other element or intervening elements may also be present. So e.g. a typical organic thin film transistor comprises a gate electrode on the substrate and a gate insulating layer on the surface of the substrate embedding the gate electrode.

In a special embodiment the substrate comprises a pattern of organic field-effect transistors, each transistor comprising:
an organic semiconductor located on the substrate;
a gate structure positioned to control the conductivity of a channel portion of the semiconductor; and
conductive source and drain electrodes located at opposite ends of the channel portion,
   wherein the organic semiconductor is at least one compound of the formula (I) or comprises at least one compound of the formula (I).

In a further special embodiment a substrate comprises a pattern of organic field-effect transistors, each transistor comprising at least one organic semiconducting compound located on the substrate forms an or is part of an integrated circuit, wherein at least part of the transistors comprise at least one compound of the formula (I) as semiconducting compound. Preferably, all of the transistors comprise at least one compound of the formula (I) as semiconducting compound.

Any material suitable for the production of semiconductor devices can be used as the substrate. Suitable substrates include, for example, metals (preferably metals of groups 8, 9, 10 or 11 of the periodic table, e.g. Au, Ag, Cu), oxidic materials (like glass, quartz, ceramics, SiO₂), semiconductors (e.g. doped Si, doped Ge), metal alloys (e.g. on the basis of Au, Ag, Cu, etc.), semiconductor alloys, polymers (e.g. polyvinylchloride, polyolefines, like polyethylene and polypropylene, polyesters, fluoropolymers, polyamides, polyurethanes, polyalkyl(meth)acrylates, polystyrene and mixtures and composites thereof, inorganic solids (e.g. ammonium chloride), and combinations thereof. The substrate can be a flexible or inflexible solid substrate with a curved or planar geometry, depending on the requirements of the desired application.

A typical substrate for semiconductor devices comprises a matrix (e.g. quartz or polymer matrix) and, optionally, a dielectric top layer (e.g. SiO₂). The substrate also may include electrodes, such as the gate, drain and source electrodes of the OFETs which are usually located on the substrate (e.g. deposited on the nonconductive surface of the dielectric top layer). The substrate also includes conductive gate electrodes of the OFETs that are typically located below the dielectric top layer (i.e., the gate dielectric).

According to a special embodiment, a gate insulating layer is formed on a part of the surface of the substrate or on the entire surface of the substrate including the gate electrode(s). Typical gate insulating layers comprise an insulating substance, preferably selected from inorganic insulating substances such as SiO₂, SiN, etc., ferroelectric insulating substances such as Al₂O₃, Ta₂O₅, La₂O₅, TiO₂, Y₂O₃, etc organic insulating substances such as polyimides, benzocyclobutene (BCB), polyvinyl alcohols, polyacrylates, etc. and combinations thereof.

Source and drain electrodes are located on the surface of the substrate at a suitable space from each other and the gate electrode with the copper semiconducting compound, at least one compound of the formula (I) being in contact with source and drain electrode, thus forming a channel.

Suitable materials for source and drain electrodes are in principal, any electrically conductive materials. Suitable materials include metals, preferably metals of groups 8, 9, 10 or 11 of the periodic table, e.g. Pd, Au, Ag, Cu, Al, Ni, Cr, etc. Preferred electrically conductive materials have a resistivity lower than about 10⁻³, more preferably lower than about 10⁻⁴, and most preferably lower than about 10⁻⁶ or 10⁻⁷ ohm metres.

According to a special embodiment, the drain and source electrodes are deposited partially on the organic semiconductor rather than only on the substrate. Of course, the substrate can contain further components that are usually employed in semiconductor devices or ICs, such as insulators, resistive structures, capacitive structures, metal tracks, etc.

### Step b)

The application of at least one compound of the formula (I) (and optionally further semiconducting compounds) can be carried out by known methods. Suitable are lithographic techniques, offset printing, flexo printing, etching, inkjet printing, electrophotography, physical vapor transport/deposition (PVT/PVD), chemical vapor deposition, laser transfer, dropcasting, etc.

In a preferred embodiment, the compound of the formula (I) (and optionally further semiconducting compounds) is applied to the substrate by physical vapor deposition (PVD). Physical vapor transport (PVT) and physical vapor deposition (PVD) are vaporisation/coating techniques involving transfer of material on an atomic level. PVD processes are carried out under vacuum conditions and involve the following steps:
Evaporation
Transportation
Deposition

The process is similar to chemical vapour deposition (CVD) except that CVD is a chemical process wherein the substrate is exposed to one or more volatile precursors, which react and/or decompose on the substrate surface to produce the desired deposit. It was surprisingly found that compounds of the formula I can be subjected to a PVD essentially without decomposition and/or the formation of undesired by-products. The deposited material is obtained in high purity and in the form of crystals or contains a high crystalline amount. The deposited material is obtained in high homogeneity and a size suitable for use as n-type semiconductors. Generally, for physical vapor deposition, a solid source material of at least one compound of the formula (I) is heated above its vaporization temperature and the vapor allowed to deposit on the substrate by cooling below the crystallization temperature of the compound of the formula (I). The temperature of the substrate material during the deposition should be less than the temperature corresponding to the vapor pressure. The deposition temperature is preferably from 20 to 250° C., more preferably from 50 to 200° C. It was surprisingly found, that it is advantageous to increase the temperature of the substrate during deposition, (e.g. for formation of a film). In general, the higher the temperature during deposition, the higher the intensity of the diffraction peaks obtained by X-ray diffraction (XRD) of the obtained semiconducting material, the larger the grain sizes, and as a result the higher the charge carrier mobility.

The obtained semiconducting layer in general should have a thickness sufficient for ohmic contact between source and drain electrode.

The deposition can be carried out under inert atmosphere, e.g. under nitrogen, argon or helium atmosphere.

The deposition can be carried out under ambient pressure or reduced pressure. A suitable pressure range is from about 0.0001 to 1.5 bar.

Preferably, the compound of the formula (I) is applied to the substrate in a layer, having an average thickness of from 10 to 1000 nm, preferably of from 15 to 250 nm.

Preferably, the compound of the formula (I) is applied in at least partly crystalline form. In a first embodiment, the compound of the formula (I) can be employed in form of preformed crystals or a semiconductor composition comprising crystals. In a second embodiment, the compound of the formula (I) is applied by a method that allows the formation of an at least partly crystallographically ordered layer on the substrate. Suitable application techniques that allow the formation of an at least partly crystalline semiconductor layer on the substrate are sublimation techniques, e.g. the aforementioned physical vapor deposition.

According to a preferred embodiment, the applied compound of the formula (I) comprises crystallites or consists of crystallites. For the purpose of the invention, the term “crystallite” refers to small single crystals with maximum dimensions of 5 millimeters. Exemplary crystallites have maximum dimensions of 1 mm or less and preferably have smaller dimensions (frequently less than 500 μm, in particular less than 200 μm, for example in the range of 0.01 to 150 μm, preferably in the range of 0.05 to 100 μm), so that such crystallites can form fine patterns on the substrate. Here, an individual crystallite has a single crystalline domain, but the domains may include one or more cracks, provided that the cracks do not separate the crystallite into more than one crystalline domain.

The stated particle sizes of the crystals of the compounds of the formula (I), the crystallographic properties and the crystalline amount of the applied compounds can be determined by direct X-ray analysis. During the pretreatment and/or the application of the compound of the formula (I), prefereably appropriate conditions e.g. pretreatment of the substrate, temperature, evaporation rate etc. are employed to obtain films having high crystallinity and large grains.

The crystalline particles of the compounds of the formula (I) may be of regular or irregular shape. For example, the particles can be present in spherical or virtually spherical form or in the form of needles. Preferably the applied compound of the formula (I) comprises crystalline particles with a length/width ratio (L/A) of at least 1.05, more preferably of at least 1.5, especially of at least 3.

Organic field-effect transistors (OFETs), wherein the channel is made of an at least partly crystallographically ordered compound of the formula (I) as organic semiconductor material will typically have greater mobility than a channel made of non-crystalline semiconductor. Larger grains and correspondingly less grain boundaries result in a higher charge carrier mobility.

Preformed organic semiconductor crystals in general and especially crystallites can also be obtained by sublimation of the compound of the formula (I) prior to application. A preferred method makes use of physical vapor transport/deposition (PVT/PVD) as defined in more detail in the following. Suitable methods are described by R. A. Laudise et al in “Physical vapor growth of organic semiconductors” Journal of Crystal Growth 187 (1998) pages 449-454 and in “Physical vapor growth of centimeter-sized crystals of α-hexathiophene” Journal of Crystal Growth 182 (1997) pages 416-427. Both of these articles by Laudise et al are incorporated herein in their entirety by reference. The methods described by Laudise et al include passing an inert gas over an organic semiconductor substrate that is maintained at a temperature high enough that the organic semiconductor evaporates. The methods described by Laudise et al also include cooling down the gas saturated with organic semiconductor to cause an organic semiconductor crystallite to condense spontaneously.

According to a preferred embodiment, the organic field-effect transistor according to the invention is a thin film transistor. As mentioned before, a TFT has a thin film structure in which a source electrode and a drain electrode are formed on a semiconductor film layer, and an insulating film is formed if necessary. The source and drain electrode materials generally should be in ohmic contact with the semiconductor film.

In a preferred embodiment, the method according to the invention comprises the step of depositing on the surface of the substrate at least one compound (C1) capable of binding to the surface of the substrate and of binding at least one compound of the formula (I). A first aspect is a method, wherein a part or the complete surface of the substrate is treated with at least one compound (C1) to obtain a modification of the surface and allow for an improved application of the compounds of the formula (I) (and optionally further semiconducting compounds). A further aspect is a method for patterning the surface of a substrate with at least one compound of the formula (I) (and optionally further semiconducting compounds). According to this aspect, a substrate with a surface has a preselected pattern of deposition sites or nonbinding sites located thereupon is preferably used. The deposition sites can be formed from any material that allows selective deposition on the surface of the substrate. Suitable compounds are the compounds C1 mentioned below. Again, PVD can be used for the application of the compounds of the formula (I) to the substrate.

A special embodiment of step b) of the method according to the invention comprises:
depositing on areas of the surface of the substrate where a gate structure, a source electrode and a drain electrode are located at least one compound (C1) capable of binding to the surface of the substrate and of binding at least one compound of the formula (I), and
applying at least one compound of the formula (I) to the surface of the substrate to enable at least a portion of the applied compound of the formula (I) to bind to the areas of the surface of the substrate modified with (C1).
The free surface areas of the substrate obtained after deposition of (C1) can be left unmodified or be coated, e.g. with at least one compound (C2) capable of binding to the surface of the substrate and to prevent the binding of at least one compound of the formula (I).

A further special embodiment of step b) of the method according to the invention comprises:
depositing on areas of the surface of the substrate where no gate structure, a source electrode and a drain electrode are located at least one compound (C2) capable of binding to the surface of the substrate and preventing the binding of at least one compound of the formula (I), and
applying at least one compound of the formula (I) to the surface of the substrate to enable at least a portion of the applied compound to bind to the areas of the surface of the substrate not modified with (C2).

The free surface areas of the substrate obtained after deposition of (C2) can be left unmodified or be coated, e.g. with at least one compound (C1) capable of binding to the surface of the substrate and of binding at least one compound of the formula (I).

For the purpose of the present application, the term “binding” is understood in a broad sense. This covers every kind of binding interaction between a compound (C1) and/or a compound (C2) and the surface of the substrate and every kind of binding interaction between a compound (C1) and at least one compound of the formula (I), respectively. The types of binding interaction include the formation of chemical bonds (covalent bonds), ionic bonds, coordinative interactions, solvatophobic interaction, Van der Waals interactions (e.g. dipole dipole interactions), etc. and combinations thereof. In one preferred embodiment, the binding interactions between the compound (C1) and the compound of the formula (I) is a non-covalent interaction.

Suitable compounds (C2) are compounds with a lower affinity to the compounds of the formula (I) than the untreated substrate or, if present, (C1). If a substrate is only coated with at least one compound (C2), it is critical that the strength of the binding interaction of (C2) and the substrate with the compound of the formula (I) differs to a sufficient degree so that the compound of the formula (I) is essentially deposited on substrate areas not patterned with (C2). If a substrate is coated with at least one compound (C1) and at least one compound (C2), it is critical that the strength of the binding interaction of (C1) and (C2) with the compound of the formula (I) differs to a sufficient degree so that the compound of the formula (I) is essentially deposited on substrate areas patterned with (C1). In a preferred embodiment the interaction between (C2) and the compound of the formula (I) is a repulsive interaction. For the purpose of the present application, the term “repulsive interaction” is understood in a broad sense and covers every kind of interaction that prevents deposition of the crystalline compound on areas of the substrate patterned with compound (C2).

In a first preferred embodiment, the compound (C1) is bound to the surface of the substrate and/or to the compound of the formula I via covalent interactions. According to this embodiment, the compound (C1) comprises at least one functional group, capable of reaction with a complementary functional group of the substrate and/or the compound of the formula (I).

In a second preferred embodiment the compound (C1) is bound to the surface of the substrate and/or to the compound of the formula (I) via ionic interactions. According to this embodiment, the compound (C1) comprises at least one functional group capable of ionic interaction with the surface of the substrate and/or a compound of the formula (I).

In a third preferred embodiment the compound (C1) is bound to the surface of the substrate and/or to the at least one compound of the formula (I) via dipole interactions, e.g. Van der Waals forces.

The interaction between (C1) and the substrate and/or between (C1) and the compounds of the formula (I) is preferably an attractive hydrophilic-hydrophilic interaction or attractive hydrophobic-hydrophobic interaction. Hydrophilic-hydrophilic interaction and hydrophobic-hydrophobic interaction can comprise, among other things, the formation of ion pairs or hydrogen bonds and may involve further van der Waals forces. Hydrophilicity or hydrophobicity is determined by affinity to water. Predominantly hydrophilic compounds or material surfaces have a high level of interaction with water and generally with other hydrophilic compounds or material surfaces, whereas predominantly hydrophobic compounds or materials are not wetted or only slightly wetted by water and aqueous liquids. A suitable measure for assessing the hydrophilic/hydrophobic properties of the surface of a substrate is the measurement of the contact angle of water on the respective surface. According to the general definition, a “hydrophobic surface” is a surface on which the contact angle of water is >90°. A “hydrophilic surface” is a surface on which the contact angle with water is <90°. Compounds or material surfaces modified with hydrophilic groups have a smaller contact angle than the unmodified compound or materials. Compounds or material surfaces modified with hydrophobic groups have a larger contact angle than the unmodified compounds or materials.

Suitable hydrophilic groups for the compounds (C1) (as well as (C2)) are those selected from ionogenic, ionic, and non-ionic hydrophilic groups. lonogenic or ionic groups are preferably carboxylic acid groups, sulfonic acid groups, nitrogen-containing groups (amines), carboxylate groups, sulfonate groups, and/or quaternized or protonated nitrogen-containing groups. Suitable non-ionic hydrophilic groups are e.g. polyalkylene oxide groups. Suitable hydrophobic groups for the compounds (C1) (as well as (C2)) are those selected from the aforementioned hydrocarbon groups. These are preferably alkyl, alkenyl, cycloalkyl, or aryl radicals, which can be optionally substituted, e.g. by 1, 2, 3, 4, 5 or more than 5 fluorine atoms.

In order to modify the surface of the substrate with a plethora of functional groups it can be activated with acids or bases. Further, the surface of the substrate can be activated by oxidation, irradiation with electron beams or by plasma treatment. Further, substances comprising functional groups can be applied to the surface of the substrate via chemical vapor deposition (CVD).

Suitable functional groups for interaction with the substrate include:
silanes, phosphonic acids, carboxylic acids, and hydroxamic acids: Suitable compounds (C1) comprising a silane group are alkyltrichlorosilanes, such as n-(octadecyl)trichlorosilane; compounds with trialkoxysilane groups, e.g. alkyltrialkoxysilanes, like n-octadecyl trimethoxysilane, n-octadecyl triethoxysilane, n-octadecyl tri-(n-propyl)oxysilane, n-octadecyl tri-(isopropyl)oxysilane; trialkoxyaminoalkylsilanes like triethoxyaminopropylsilane and N[(3-triethoxysilyl)-propyl]-ethylen-diamine; trialkoxyalkyl-3-glycidylethersilanes such as triethoxypropyl-3-glycidylethersilane; trialkoxyallylsilanes such as allyltrimethoxysilane; trialkoxy(isocyanatoalkyl)silanes; trialkoxysilyl(meth)acryloxyalkanes and trialkoxysilyl(meth)acrylamidoalkanes, such as 1-triethoxysilyl-3-acryloxypropan. (These groups are preferably employed to bind to metal oxide surfaces such as silicon dioxide, aluminium oxide, indium zinc oxide, indium tin oxide and nickel oxide.),
amines, phosphines and sulfur containing functional groups, especially thiols: (These groups are preferably employed to bind to metal substrates such as gold, silver, palladium, platinum and copper and to semiconductor surfaces such as silicon and gallium arsenide.)

In a preferred embodiment, the compound (C1) is selected from alkyltrialkoxysilanes and is in particular n-octadecyl triethoxysilane. In a further preferred embodiment, the compound (C1) is selected from hexaalkyldisilazanes and is in particular hexamethyldisilazane (HMDS). In a further preferred embodiment, the compound (C1) is selected from C₈-C₃₀-alkylthiols and is in particular hexadecane thiol. In a further preferred embodiment the compound (C1) is selected from mercaptocarboxylic acids, mercaptosulfonic acids and the alkali metal or ammonium salts thereof. Examples of these compounds are mercaptoacetic acid, 3-mercaptopropionic acid, mercaptosuccinic acid, 3-mercapto-1-propanesulfonic acid and the alkali metal or ammonium salts thereof, e.g. the sodium or potassium salts. In a further preferred embodiment the compound (C1) is selected from alkyltrichlorosilanes, and is in particular n-(octadecyl)trichlorosilane.

Additionally to or as an alternative to deposition of said compound (C1) on the substrate, the substrate can be contacted with at least one compound (C2) capable of binding to the surface of the substrate as well as of interaction with the compound of the formula (I) to prevent deposition of (S) on areas of the substrate not patterned with compound (C1). According to a suitable embodiment, the compounds (C2) are selected from compounds with a repulsive hydrophilic-hydrophobic interaction with (S). The compounds of the formula (I) can be purified by recrystallization or by column chromatography. Suitable solvents for column chromatography are e.g. halogenated hydrocarbons, like methylene chloride. In an alternative embodiment, the compounds of the formula (I) can be recrystallized from sulfuric acid.

In a preferred embodiment, purification of the compound of the formula (I) can be carried out by sublimation. Preferred is a fractionated sublimation. For fractionated sublimation, the sublimation and/or the deposition of the compound is effected by using a temperature gradient. Preferably the compound of the formula (I) sublimes upon heating in flowing carrier gas. The carrier gas flows into a separation chamber. A suitable separation chamber comprises different separation zones operated at different temperatures. Preferably a so-called three-zone furnace is employed. A further suitable method and apparatus for fractionated sublimation is described in U.S. Pat. No. 4,036,594. In a further embodiment at least one compound of the formula (I) is subjected to purification and/or crystallization by physical vapor transport. Suitable PVD techniques are those mentioned before. In a physical vapor transport crystal growth, a solid source material is heated above its vaporization temperature and the vapor is allowed to crystallize by cooling below the crystallization temperature of the material. The obtained crystals can be collected and afterwards applied to specific areas of a substrate by known techniques, as mentioned above. A further aspect is a method for patterning the surface of a substrate with at least one compound of the formula (I) (and optionally further organic semiconducting compounds) by PVD. According to this aspect, a substrate with an unmodified surface, or a surface being at least partly covered with a substance that improves deposition of at least one compound of the formula (I) or a surface that has a preselected pattern of deposition sites located thereupon is preferably used. The deposition sites can be formed from any material that allows selective deposition on the surface of the substrate. Suitable compounds are the aforementioned compounds C1, which are capable of binding to the surface of the substrate and of binding at least one compound of the formula (I).

The invention will now be described in more detail on the basis of the accompanying figures and the following examples.

### EXAMPLES

I) BPE-PTCDI

BPE-PTCDI was synthesized form peryiene-3,4:9,10-tetracarboxylic acid bisanhydride and phenethylamine by known methods. The purification was carried out by three consecutive vacuum sublimations using a three-temperature-zone furnace (Lindberg/Blue Thermo Electron Corporation). The three temperature zones were set to be: 400° C., 350° C. and 300° C. and the vacuum level during sublimation was 10⁻⁶ Torr or less while the starting material was placed in the first temperature zone.

Highly doped n-type Si wafers (2.5×2.5 cm) with a thermally grown dry oxide layer (capacitance per unit area Cᵢ=10 nF/cm²) as gate dielectric were used as substrates. The substrate surfaces were cleaned with acetone followed by isopropanol. Afterwards, the surface of the substrate was left unmodified (a) or was modified with n-octadecyl trimethoxysilane (b) or hexamethyidisilazane (c):
(a) No surface treatment
(b) A few drops of n-octadecyl trimethoxysilane (C₁₈H₃₇Si(OCH₃)₃, obtained from Aldrich Chem. Co.) were deposited on top of the preheated substrate (˜100° C.) inside a vacuum desiccator. The desiccator was immediately evacuated (25 mm Hg) and the substrate left under vacuum for 5 hours. Finally, the substrates were baked at 110° C. for 15 min, rinsed with isopropanol and dried with a stream of air.
(c) Hexamethyldisilazane [(CH₃)₃—Si—N—Si—(CH₃)₃), HMDS] treatment of the substrate was performed using a Yield Enhancement System (YES-100). Afterwards, BPE-PTCDI thin films (40 nm) were vacuum-deposited on the substrates at room temperature and at elevated temperatures (i.e. 60° C., 90° C., 125° C., 150° C. and 200° C.) with a deposition rate of 1.0 Å/s at 10⁻⁶ Torr. The film thickness was determined by quartz crystal microbalance (QCM).

Top-contact devices were fabricated by depositing gold source and drain electrodes onto the organic semiconductor films through a shadow mask with channel length of 2000 μm and channel width of 200 μm. The electrical characteristics of the obtained organic thin film transistor devices were measured using a Keithley 4200-SCS semiconductor parameter analyzer. Key device parameters, such as charge carrier mobility (μ), on/off current ratio (Iₒₙ/I_{off}), were extracted from the drain-source current (I_{d})-gate voltage (V_{g}) characteristics. The morphology of BPE-PTCDI thin films was determined using an atomic force microscope (AFM) (Multimode Nanoscope III, Digital Instrument Inc.) in tapping mode. Out-of-plane x-ray diffraction (XRD) measurement was carried out with a Philips X'Pert PRO system. The beam wavelength was 1.5406 Å operated at 45 KeV and 40 mA. Cyclic voltammetry data were obtained from a saturated solution in anhydrous methylene chloride under argon with 0.1 M tetrabutyl ammonium hexafluorophosphate as supporting electrolyte. The scan rate was 50 mVs⁻¹. A silver wire was used as pseudoreference electrode. The ferrocene/ferrocenium redox couple was used as reference (Fc/Fc+E_{1/2}=0.56 V in the used system).

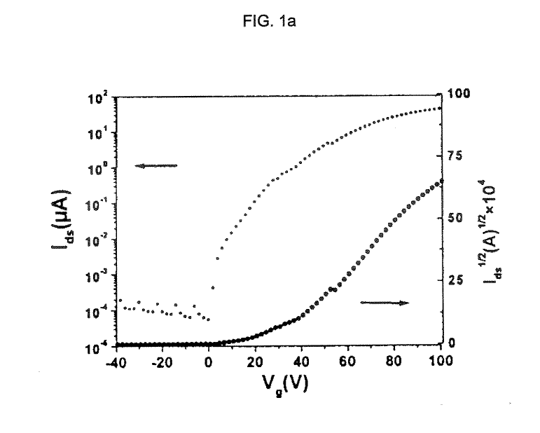

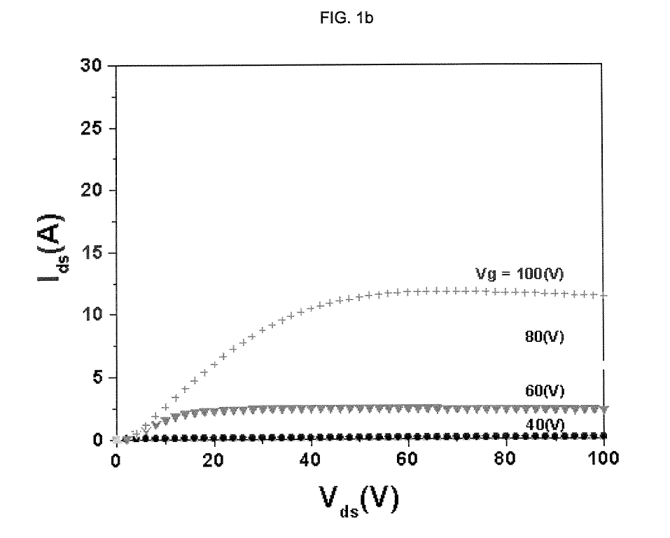

FIG. 1(a) shows the current-voltage characteristic (I_{ds}−V_{g} for V_{ds}=100 V) of a BPE-PTCDI TFT. left axis, symbols on the left: log scale; right axis, symbols on the right: regular scale

Typical current-voltage characteristics (I_{ds}−V_{ds} for various V_{g}) of a BPE-PTCDI TFT are shown in FIG. 1(b).

The following table 1 gives a summary of average field effect mobilities (cm²/Vs) over at least five devices, on/off ratio and treshhold voltage for BPE-PTCDI, deposited at various substrate temperatures.

TABLE 1

| Substrate | | | | |
|---|---|---|---|---|
| temperature | Surface | Mobility | | Vₜₕ |
| [° C.] | treatment | [cm²/Vs] | on/off ratio | [V] |
| 25 | without | 2.0 × 10⁻⁵ | 1.8 × 10³ | 24 |
| 25 | OTS | 0.03 | 2.2 × 10⁶ | 11 |
| 25 | HMDS | 0.02 | 2.9 × 10⁵ | 44 |
| 90 | without | 4.0 × 10⁻⁵ | 2.3 × 10² | 28 |
| 90 | OTS | 0.04 | 2.3 × 10³ | 38 |
| 90 | HMDS | 0.03 | 9.1 × 10² | 37 |
| 125 | without | 0.02 | 1.4 × 10⁴ | 36 |
| 125 | OTS | 0.08 | 8.9 × 10⁴ | 18 |
| 125 | HMDS | 0.06 | 6.4 × 10⁴ | 29 |
| 150 | without | 0.03 | 6.1 × 10² | 38 |
| 150 | OTS | 0.11 | 3.3 × 10⁵ | 29 |
| 150 | HMDS | 0.07 | 1.9 × 10⁵ | 30 |

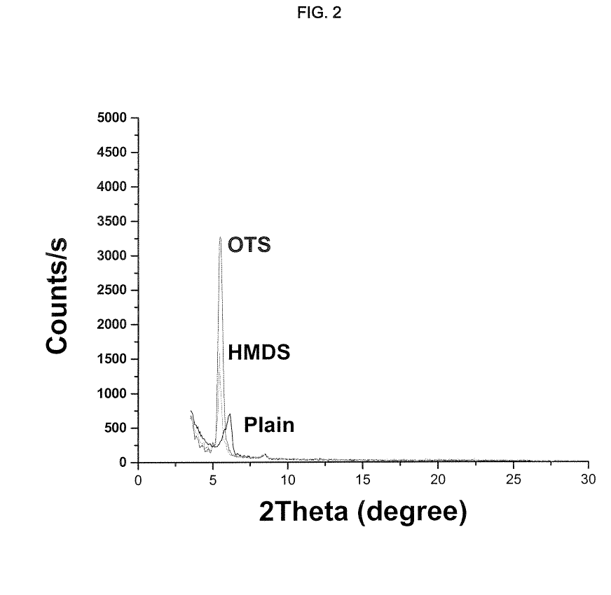

The out-of-plane XRD patterns of 40 nm BPE-PTCDI thin film deposited at a temperature of 150° C. on a plain substrate and substrates where the surface was treated with n-(octadecyl)trimethoxysilane (OTS) and hexamethyidisilazane (HMDS) are shown in FIG. 2. The lattice spacing is 1.42 nm, which is very close to half the molecular length of the long axis of the molecule. This indicates that the BPE-PTCDI molecules adapt an edge-on conformation in thin films. A general trend is that, the higher the substrate temperature during thin film deposition, the higher the intensity of the diffraction peak, consistent with the observation of larger grain sizes and as a result higher charge carrier mobilities.

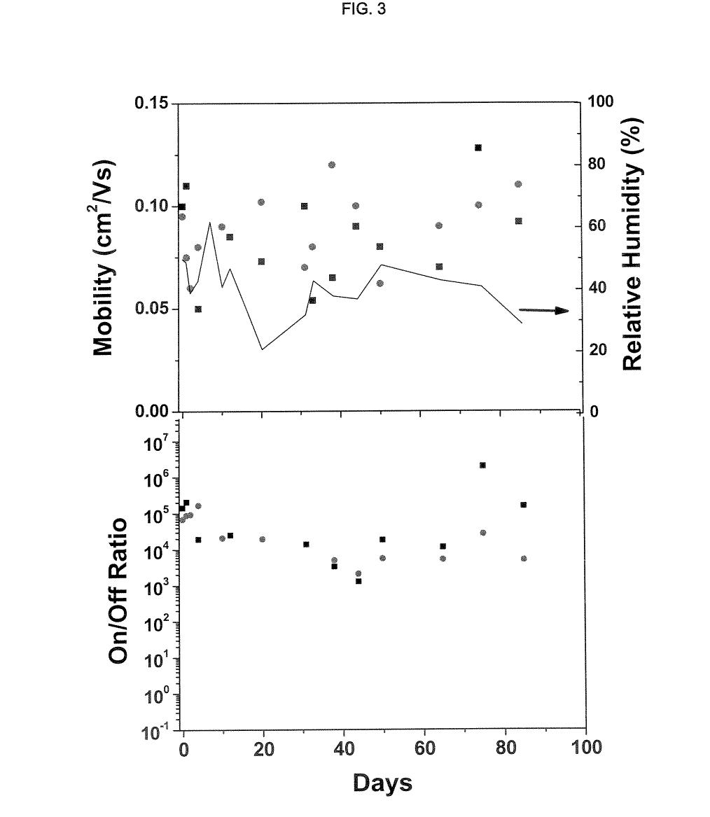

Air-stability measurements of BPE-PTCDI TFTs are shown in FIG. 3.

FIG. 3(a), left axis: charge carrier mobility (dots: exposed to air only; squares: exposed to air and ambient light), right axis: relative humidity (curve)

FIG. 3(b): on/off ratio

Air-stability measurements were carried out by monitoring the charge carrier mobility (FIG. 3a) and on/off ratio (FIG. 3b) as a function of time. (dots: only exposed only to air, squares: exposed to air and ambient light). All electrical tests were performed in air under environment conditions. The devices did not show a significant decrease of the initial values. This shows that BPE-PTCDI is an air-stable n-type semiconductor with good application properties.

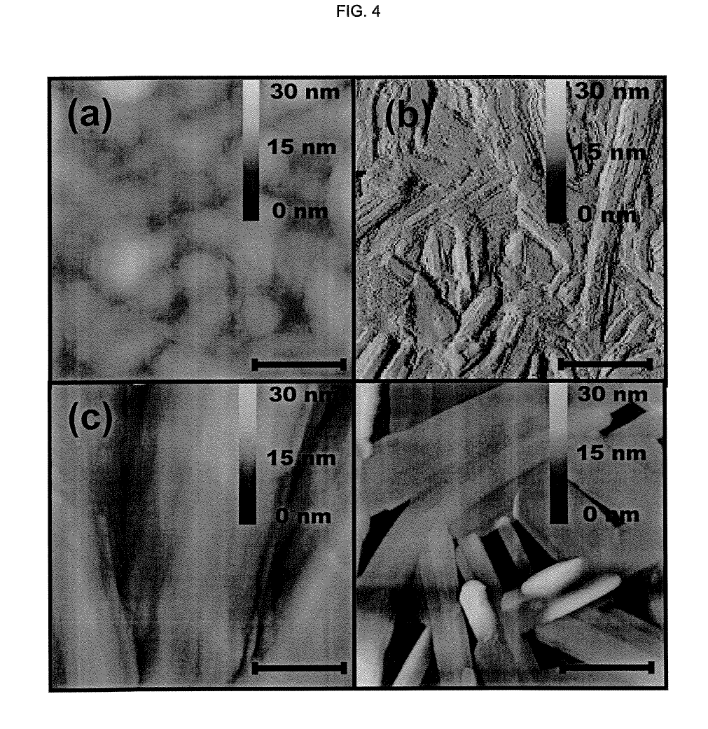

FIG. 4 shows the atomic force microscope (AFM) images of 45 nm BPE-PTCDI thin films on substrates treated with n-(octadecyl)trimethoxysilane for various substrate temperatures (room temperature, 125° C., 150° C. and 200° C.) during thin film deposition. The grain size becomes larger as the substrate temperature increases, which may be responsible for the increase in mobility with the substrate temperature during deposition.

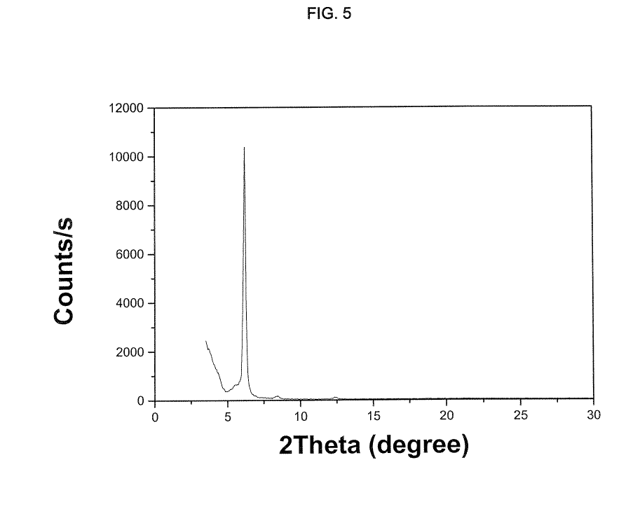

FIG. 5 shows the out-of-plane XRD patterns of 40 nm BPE-PTCDI thin film deposited at a temperature of 125° C. on a substrates where the surface was treated with n-(octadecyl)trimethoxysilane (OTS).

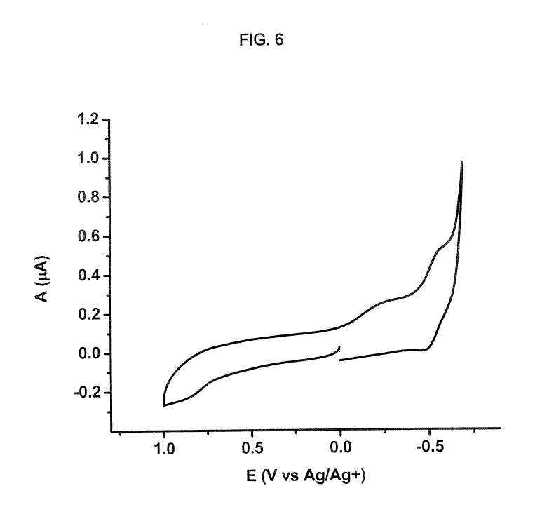

FIG. 6 shows the reduction potential of BPE-PTCDI measured by cyclic voltammetry. The LUMO level was calculated using the onset of the reduction peak according to methods known from the literature (D. M. de Leeuw, M. M. J. Simenon, A. R. Brown, R. E. F. Einerhand, Synth. Met. 1997, 87, 53). With the ferrocene/ferrocenium redox couple as reference a LUMO of −4.1 eV was determined, which is high in comparison with further air stable organic semiconductors known from the art, such as dicyano-substituted perylene-3,4,9,10-tetracarboxylic diimide (−4.3 to −4.6 eV).

Use of BPE-PTCDI in inverters: BPE-PTCDI and pentacene were purified by three consecutive vacuum sublimations using a three-temperature-zone furnace (Lindberg/Blue Thermo Electron Corporation) under high vacuum (less than 5×10⁻⁶ Torr). The starting material was placed in the first temperature zone. The three temperature zones were set to be 400° C., 350° C. and 300° C. for BPE-PTCDI and 249° C., 160° C. and 100° C. for pentacene, respectively. A highly doped n⁺⁺ silicon substrate was used as a common gate electrode. A thermally grown silicon dioxide (300 nm, capacitance Cᵢ=10 nF/cm²) was used as the dielectric layer. The substrates were cleaned by rinsing with acetone followed by isopropyl alcohol and then treated with octadecyl-trimethoxysilane (C₁₈H₃₇Si(OCH₃)₃, OTS). A few drops of pure OTS were loaded on top of a preheated quartz block (˜100° C.) inside a vacuum desiccator. The desiccator was immediately evacuated (−25 mmHg) and the SiO₂/Si substrate was treated with the OTS to give a hydrophobic surface. Finally, the substrates were then baked at 110° C. for 15 min, rinsed with isopropanol and dried with a stream of air. For the production of top contact n-type transistors a BPE-PTCDI layer (45 nm thickness) was deposited on top of the substrates at a pressure less than 2×10⁻⁶ torr with a deposition rate of 1.0 Å/s using a vacuum thin-film deposition system (Angstrom Engineering, Inc., Canada). The substrates were held at about 200° C. during thin film deposition. Elevated substrate temperature was found to lead to larger grain size and thus higher charge carrier mobilities. The area for the n-type film is about 1 cm by 2 cm. The rest of the area was covered by a thin glass mask during the film deposition of the p-type semiconductor. For the production of top contact p-type transistors, a pentacene layer (45 nm thickness) was deposited on top of the substrates at a pressure less than 2×10⁻⁶ torr with a deposition rate of 1.0 Å/s while covering the thin films of perylene derivatives that had been already deposited. The substrates were held at 60° C. during thin film deposition. Shadow masks with various channel length (L) and width (W) were used for gold (ca. 40 nm) metal evaporation to make both p-type and n-type top-contact thin film transistors. In order to match the source/drain current from both types of transistors to achieve optimum operation conditions for the inverters, W/L of 10 (i.e., W/L=2000 μm/200 μm) and 50 (ie., W/L=2500 μm/50 μm) were used for p-type and n-type transistors, respectively. To form an inverter, both the drain electrodes from each of the p-type and n-type transistors were connected using an aluminum wire with both of its ends attached to the gold electrodes with a soft metal such as Indium.

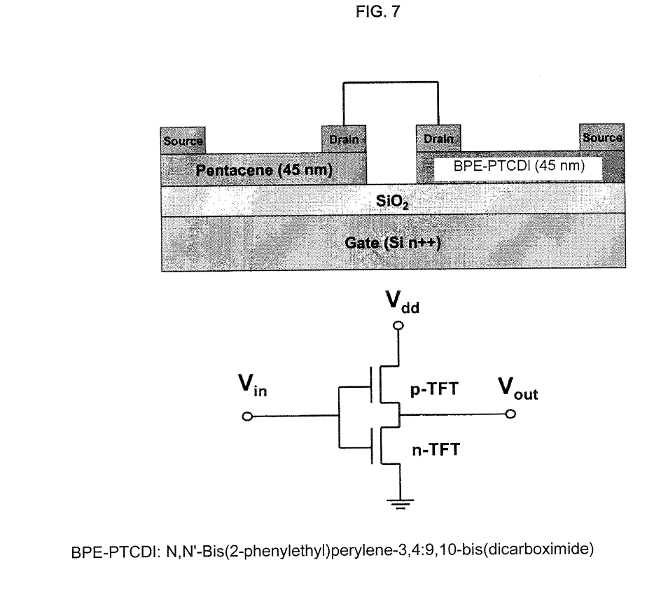

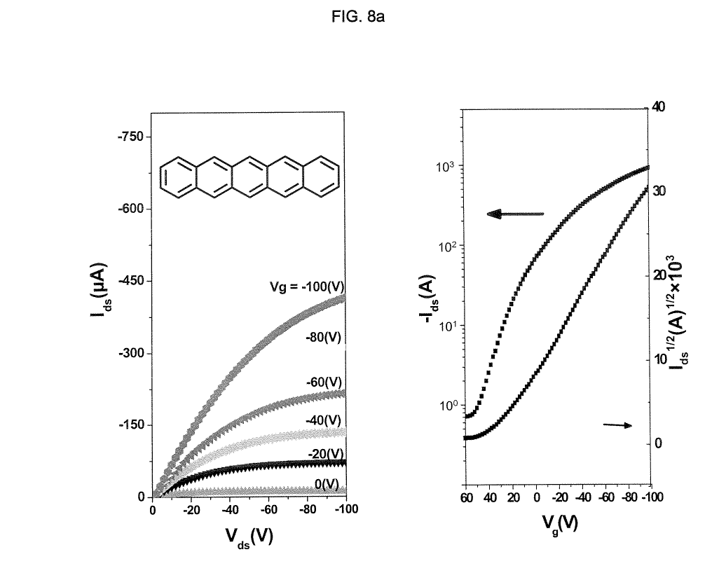

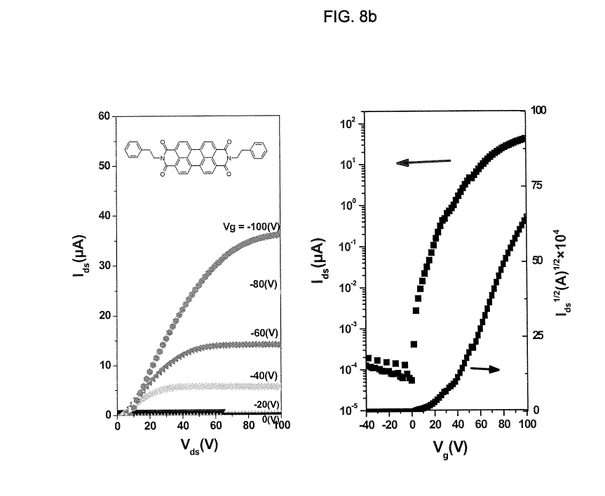

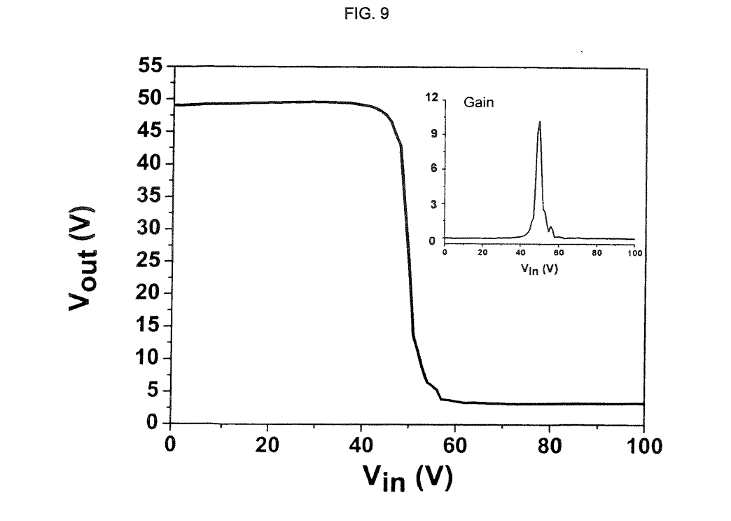

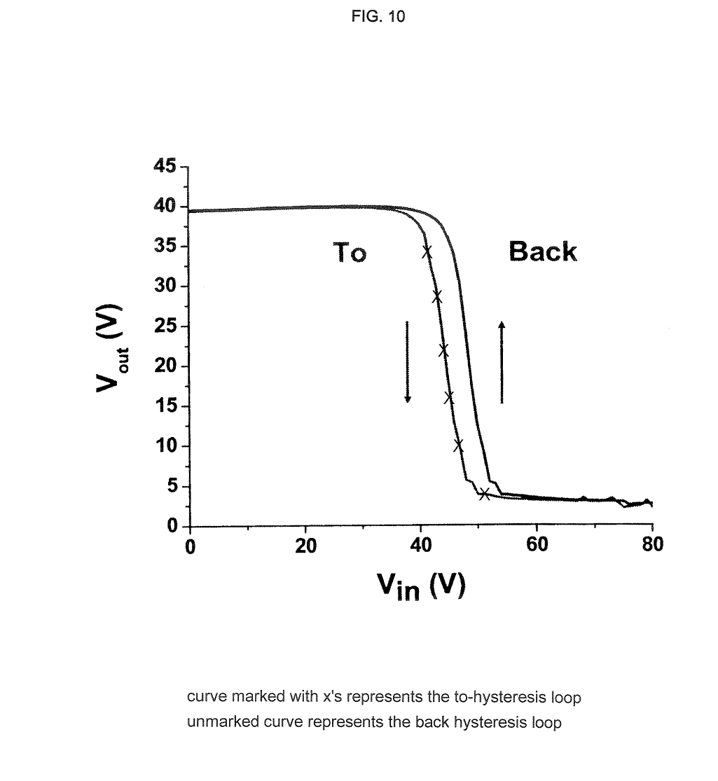

The final inverter structure is shown in FIG. 7. OTFTs with a W/L ratio of 20 were made as references. The electrical characteristics of OTFT devices and the corresponding inverters were measured using a Keithley 4200-SCS semiconductor parameter analyzer in ambient lab environment. Key device parameters for transistors such as charge carrier mobilities were extracted from the drain-source current (I_{d})-gate voltage (V_{g}) characteristics. Parameters for the inverter such as gain, noise margin and output voltage swing were extracted from the transfer curves of output voltage (Vₒᵤₜ) vs. input voltage (Vᵢₙ). Typical current-voltage characteristics of pentacene and BPE-PTCDI are shown in FIGS. 8(a) and 8(b). The extracted mobilities for pentacene TFTs were around 0.5 cm²Vs. The on/off ratio was 1.2×10⁵ and the threshold voltage was −8.7 V. The n-type mobilities, on/off ratio and threshold voltage for the BPE-PTCDI were 0.12 cm²/Vs, 2.2×10⁵, 28.3 V. The excellent air-stability of both the p-type and n-type materials enables the organic TFTs to work very well in ambient air. As shown in FIG. 9, for V_{dd}=50 V, the highest gain for BPE-PTCDI inverter is about 10.5, the noise margin is 8.5 V and the output voltage swing is about 46 V. Here the output voltage swing is defined as the difference between the maximum and minimum values of the output voltage. The corresponding values are 5.5, 4.4 V, and 26 V for V_{dd}=30 V, and 6.5, 6 V, and 35 V for V_{dd}=40 V. The output voltage starts from values close to the applied voltage V_{dd}, and then dramatically drops to very low values. The hysteresis is shown in FIG. 10. Minor hysteresis was observed and there could be several causes for it. Both mobile charges in the gate dielectric, charge trapping at the dielectric/semiconductor interface, and/or imperfect coupling between the p- and n-channel transistors could lead to hysteresis. We did not observe any hysteresis for pentacene transistors while the n-channel transistors operating at V_{ds} of 40V and 50V exhibit very small but observable hysteresis, possibly due to charge trapping at the semiconductor/insulator interface.

II) N,N′-dimethylperylene-3,4,9,10-tetracarboxylic diimide (DME-PTCDI)

DME-PTCDI was purified by three consecutive vacuum sublimations using a three-temperature-zone furnace (Lindberg/Blue Thermo Electron Corporation). The material used was collected from the second temperature zone (T2) after the third purification.

TABLE 2
Electrical characteristics

| Substrate | | | | |
|---|---|---|---|---|
| temperature | Surface | Mobility | | Vₜ |
| [° C.] | treatment | [cm²/Vs] | on/off ratio | [V] |
| 25 | without | — | — | — |
| 25 | OTS | 0.0007 | 50290 | 15 |
| 25 | HMDS | 0.0002 | 21067 | 5 |
| 125 | without | 0.003 | 8198 | −1.87 |
| 125 | OTS | 0.037 | 22924 | 36 |
| 125 | HMDS | 0.016 | 2442 | 52 |

What is claimed is:

1. A method for producing an organic field-effect transistor, comprising the steps of:
a) providing a substrate comprising a gate structure, a source electrode and a drain electrode located on the substrate, and
b) applying at least one compound of the formula I 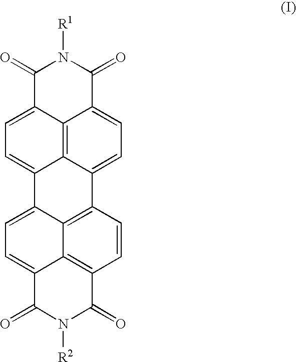
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound to the area of the substrate where the gate structure, the source electrode and the drain electrode are located.

2. The method as claimed in claim 1, wherein in the formula I n is 1 or 2.

3. The method as claimed in claim 1, wherein in the formula I, R^{a} and R^{b} are selected from 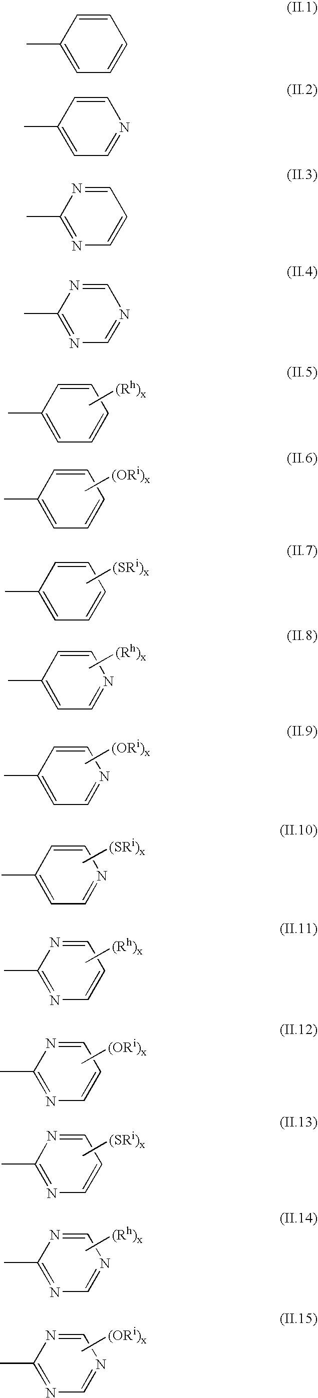 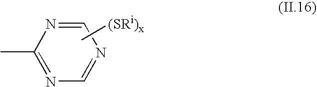
wherein
the residues R^{h} in formulae II.5, II.8, II.11 and II.14 are selected independently of one another from C₁-C₃-alkyl, C₁-C₃-fluoroalkyl, fluorine, chlorine, bromine, NE¹E², nitro and cyano, where E¹ und E², independently of one another, are hydrogen, alkyl, cycloalkyl, heterocycloalkyl, aryl or hetaryl,
the residues Rⁱ in formulae II.6, II.7, II.9, II.10, II.12, II.13, II.15 and II.16 are selected independently of one another from C₁-C₃-alkyl,
x in formulae II.5, II.6 and II.7 is 1, 2, 3, 4 or 5,
in formulae II.8, II.9 and II.10 is 1, 2, 3 or 4,
in formulae II.11, II.12 and II.13 is 1, 2 or 3,
in formulae II.14, II.15 and II.16 is 1 or 2.

4. The method as claimed in claim 1, wherein a compound of the formula 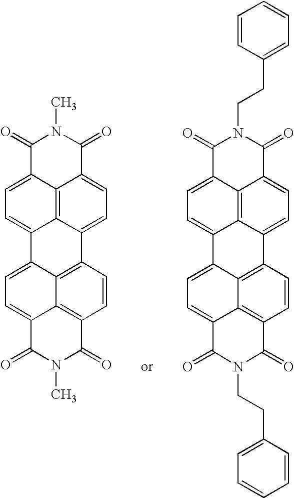
is employed as n-type organic semiconducting compound.

5. The method as claimed in claim 1, wherein the compound of the formula I is applied to the substrate by physical vapor deposition.

6. The method as claimed in claim 5, wherein the temperature of the substrate material during the deposition is less than the temperature corresponding to the vapor pressure.

7. The method as claimed in claim 5, wherein the temperature of the substrate material during the deposition is in the range of from 20 to 250° C., preferably in the range of from 50 to 200° C.

8. The method as claimed in claim 5, wherein the compound of the formula I is applied to the substrate in a layer, having an average thickness of from 10 to 1000 nm, preferably of from 15 to 350 nm.

9. The method as claimed in claim 1, wherein the compound of the formula I is applied in at least partly crystalline form.

10. The method as claimed in claim 1, wherein the compound of the formula I is applied to the substrate in form of a thin film.

11. The method as claimed in claim 1, comprising the step of depositing on the surface of the substrate at least one compound (C1) capable of binding to the surface of the substrate and of binding at least one compound of the formula I.

12. The method as claimed in claim 11, wherein the compound (C1) is selected from alkyltrialkoxysilanes and is in particular n-octadecyl trimethoxysilane or n-octadecyl triethoxysilane.

13. The method as claimed in claim 11, wherein the compound (C1) is selected from hexaalkyldisilazanes and is in particular hexamethyldisilazane.

14. The method as claimed in claim 1, wherein a compound of the formula I is employed that results from purification by sublimation, physical vapor transport, recrystallization or a combination of two or more of these methods.

15. An organic field-effect transistor comprising:
a substrate,
a gate structure, a source electrode and a drain electrode located on the substrate, and
at least one compound of the formula I 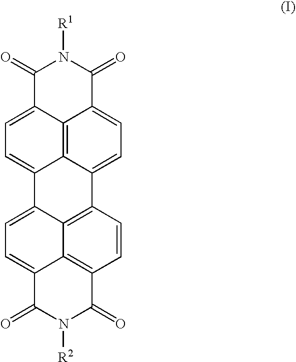
wherein,
R¹ is a (CₙH₂ₙ)—Rₐ group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound at least on the area of the substrate where the gate structure, the source electrode and the drain electrode are located.

16. The organic field-effect transistor of claim 15 in form of a thin film transistor.

17. A method for producing a substrate comprising a pattern of n-type organic field-effect transistors, wherein at least part of the transistors comprise as n-type organic semiconducting compound a compound of the formula I and are obtained by a method as defined in claim 1.

18. A substrate comprising a pattern of n-type organic field-effect transistors wherein at least part of the transistors comprise at least one compound of the formula I 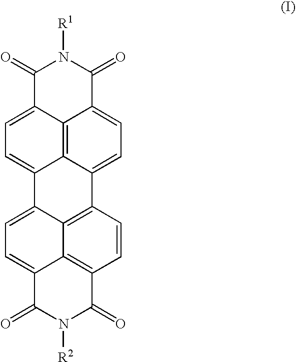
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound.

19. A method for producing an electronic device comprising the step of providing on a substrate a pattern of organic field-effect transistors, wherein at least part of the transistors comprise at least one compound of the formula I 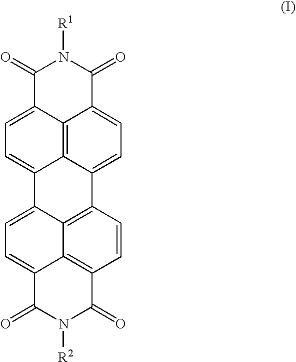
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound.

20. An electronic device comprising on a substrate a pattern of organic field-effect transistors, wherein at least part of the transistors comprise at least one compound of the formula I 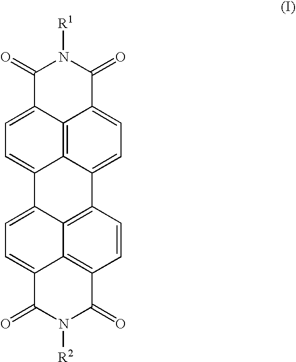
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound.

21. A method for producing a crystalline compound of the formula I 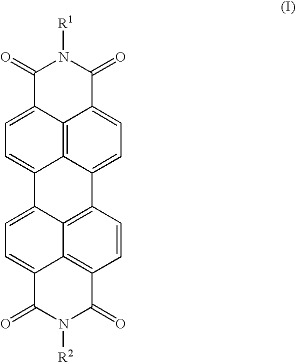
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R_{b} is hydrogen or unsubstituted or substituted cycloalkyl, bicycloalkyl, cycloalkenyl, heterocycloalkyl, aryl or hetaryl, and n is an integer of 1 to 4, comprising subjecting a compound of the formula I to a physical vapor transport.

22. An organic light-emitting diode (OLED) comprising at least one compound of the formula I 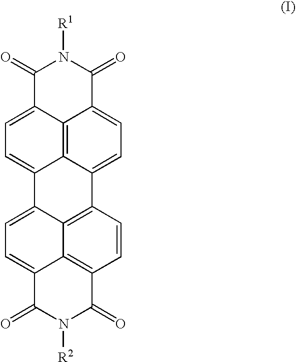
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound.

23. An inverter comprising at least one compound of the formula I 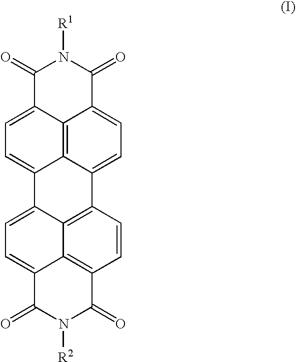
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound.

24. An organic solar cell comprising at least one compound of the formula I 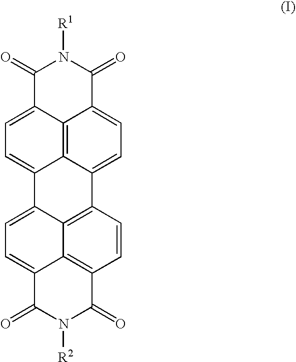
wherein,
R¹ is a (CₙH₂ₙ)—R^{a} group or a three- to five-membered saturated, unsubstituted or substituted carbocycle, wherein R^{a} is hydrogen or unsubstituted or substituted cycloalkyl, bicycloalkyl, cycloalkenyl, heterocycloalkyl, aryl or hetaryl, and n is an integer of 1 to 4,
R² is a (CₙH₂ₙ)—R^{b} group or a three- to five-membered saturated, unsubstituted or substituted carbocycle, wherein R^{b} is hydrogen or unsubstituted or substituted cycloalkyl, bicycloalkyl, cycloalkenyl, heterocycloalkyl, aryl or hetaryl, and n is an integer of 1 to 4, as n-type organic semiconducting compound.
